(19) United States
(12) Patent Application Publication
(10) Pub. No.: US 2013/0316926 A1
(43) Pub. Date: Nov. 28, 2013

(54) METHOD FOR THE MONITORING OF SMOKING CESSATION COMPLIANCE AND RECOVERY, THERAPEUTIC INTERVENTION, AND RISK MANAGEMENT

(71) Applicant: Health Diagnostic Laboratory, Inc., Richmond, VA (US)

(72) Inventor: Rebecca E. CAFFREY, N Chesterfield, VA (US)

(73) Assignee: Health Diagnostic Laboratory, Inc., Richmond, VA (US)

(21) Appl. No.: 13/903,809

(22) Filed: May 28, 2013

Related U.S. Application Data
(60) Provisional application No. 61/651,971, filed on May 25, 2012.
(60) Provisional application No. 61/786,898, filed on Mar. 15, 2013.

(51) Int. Cl. G01N 33/574; C12Q 1/68; G01N 33/68

(52) U.S. Cl. CPC G01N 33/57488; G01N 33/6893; C12Q 1/6886
USPC 506/9; 506/18; 506/15; 506/19; 506/12; 506/17

(57) ABSTRACT

The invention provides compositions and methods for determining patient compliance with a smoking cessation program, and monitoring the patient for physiological recovery from the deleterious effects of smoking, with particular emphasis on those effects that impact risk of cardiovascular disease and development of cancer. The invention also provides compositions and methods for treating a subject according to the patient compliance with the smoking cessation program.

### CROSS-REFERENCE TO RELATED APPLICATIONS

This application claims the benefit of priority to U.S. Provisional Patent Application Ser. No. 61/651,971, filed May 25, 2012; and U.S. Provisional Patent Application Ser. No. 61/786,898, filed Mar. 15, 2013; both of which are hereby incorporated by reference in their entirety.

### FIELD OF THE INVENTION

The invention relates to biomarker panels and behavioral modification programs for assessing patient compliance with a smoking cessation regimen.

### BACKGROUND OF THE INVENTION

According to the World Health Organization, tobacco use is the single most preventable cause of death worldwide killing more than 5 million people per year and responsible for 1 in 10 adult deaths. It is also attributable to 11% of deaths from ischaemic heart disease, the world's leading killer and responsible for more than 70% of deaths from lung, trachea and bronchus cancers. See World Health Organization—Tobacco Free Initiative Publication (2005), “Why Tobacco is a Public Health Priority?,” hereby incorporated by reference to its entirety. Smoking can also cause serious health problems, e.g. it increases the risk of cardiovascular diseases, cancer and other diseases. See U.S. Department of Health and Human Services' Publication (2010), “How Tobacco Smoke Causes Disease: The Biology and Behavioral Basis for Smoking-Attributable Disease—A Report of the Surgeon General” and National Toxicology Program Publication, “Report on Carcinogens (12^{th} Edition),” both of which are hereby incorporated by reference to their entirety. For this reason, healthcare providers urge patients to cease smoking to reduce their risk for developing any of the above-mentioned diseases and avoid a premature death.

Nicotine is the psychoactive drug in tobacco products that produces dependence. The combination of strong physical substance dependence and psychological dependence make quitting smoking difficult and often requires multiple attempts. Indeed, cigarette or tobacco users often experience relapse because of stress, weight gain and withdrawal symptoms.

Patient management during the smoking cessation process is difficult for the healthcare provider because a substantial number of patients lie to their physicians about their health habits, including their smoking habits. In a 2004 WebMD survey conducted on 1500 participants, 45% of respondents admitted to overtly lying or stretching the truth when talking to their doctors about their health habits and lifestyle. In the survey, 22% of the survey respondents admitted that they lied to their doctors about smoking, 38% lied about following the doctors' orders to take medications, and 32% lied about their diet and exercise habits. In another study of smokers who were ordered to quit by their doctors, 17% smoked while denying doing so. Among men, the percentage was 21%, and among ex-smokers, the figure was 27%. The highest value, 34%, was found among patients with chronic obstructive pulmonary disease (COPD). Therefore, accurate information about actual patient compliance with smoking cessation orders is a key component in assessing recovery and health risks at a behavioral and biochemical level.

There is thus a need in the art to develop biomarker panels and behavioral modification programs for assessing patient compliance with a smoking cessation regimen. This invention answers this need.

### SUMMARY OF THE INVENTION

This invention relates to a biomarker panel for assessing patient compliance with a smoking cessation program. The biomarker panel comprises a first analyte-binding ligand that specifically binds to cotinine, or an antigen-binding fragment thereof and a second analyte-binding ligand that specifically binds to anabasine, or an antigen-binding fragment thereof; and at least two or more of the following biomarkers: (i) one or more carcinogen biomarkers, wherein the one or more carcinogen biomarker includes 4-(methylnitrosoamino)-1-(3-pyridyl)-1-butanol (NNAL) and at least one carcinogen biomarkers, e.g., MHBMA, HPMA, HBMA, SPMA, HEMA, 1-HOP, NNAL, NNK, polycystic aromatic hydrocarbon-DNA adducts (PAH-DNA), 4-ABP-hemoglobin adducts, benzene and benzene metabolites, and OGG1 activity; (ii) one or more antioxidants, e.g., vitamin A, vitamin C, vitamin E, lutein, lycopene, and β-carotene; (iii) one or more oxidative-stress biomarkers, e.g., malondialdehyde (MDA), oxidized glutathione (GSSG), reduced glutathione (GSH), and GSSG/GSH ratio; (iv) one or more cardiovascular risk biomarkers; (v) one or more analytes associated with insulin resistance, glycemic control, and/or beta cell dysfunction; (vi) a plurality of immuno-markers from an early lung cancer detection test (EarlyCDT®); and (vii) one or more polymorphic genes involved in the risk of development of cancer or detection of presence of cancer.

In an exemplary embodiment, one or more cardiovascular risk biomarkers in the biomarker panel include low density lipoprotein particle number (LDL-P), LDL-cholesterol (LDL-C), apolipoprotein A-1 (ApoA-1), apolipoprotein B (ApoB), triglyceride, high density lipoprotein particle number (HDL-P), high density lipoprotein-cholesterol (HDL-C), high sensitivity C-reactive protein (hs-CRP), remnant-like lipoproteins (RLPs), RLP-c (cholesterol measures), lipoprotein A (apoA-I, Lp(a) or HDL), HDL2, ApoB:ApoA-1 ratio, ApoB, ApoB48, ApoE, ApoC, Lp(a) mass, Lp(a) cholesterol, large VLDL-P, small LDL-P, large HDL-P, VLDL-size, LDL size, HDL size, LP-IR score, and subclasses, genetic variants, fragments and complexes thereof.

In another exemplary embodiment, the biomarker panel includes one or more analytes associated with insulin resistance, glycemia and/or beta cell dysfunction, e.g., glucose, insulin, hemoglobin (Hb) A1c, fructosamine, mannose, D-mannose, mannose-binding lectin, 1,5-anhydroglucitol (1,5 AG), glycation gap (glycosylation gap), serum amylase, anti-GAD antibody, c-peptide, intact pro-insulin, leptin, adiponectin, ferritin, free fatty acids, lipoprotein-associated phospholipase A2 (Lp-PLA2), fibrinogen, myeloperoxidase, cystatin C, homocysteine, F2-isoprostanes, α-hydroxybutyrate (AHB), linoleoyl glycerophosphocholine (GPC), oleic acid, analytes associated with IR score, analytes associated with HOMA (Homeostasis Model Assessment) IR score, analytes associated with CLIX score, gamma-glutamic transferase (GGT), uric acid, vitamin B12, homocysteine, 25-hydroxyvitamin D, TSH, earned glomerular filtration rate, and serum creatinine.

Cancer polymorphic genes, according to another embodiment of this invention, include variegation 3-9 homolog 2 (SUV39H2) polymorphism, CRP gene polymorphism, and genetic polymorphisms for DNA repair enzymes.

The biomarker panel, described herein, includes various embodiments, for example, (a) at least two or more of the biomarkers in the biomarker panel comprise the biomarkers in (i), (iv), (v), and (vi); (b) at least two or more of the biomarkers in the biomarker panel comprise the biomarkers in (i), (iv), and (v); (c) at least two or more of the biomarkers in the biomarker panel comprise the biomarkers in (i), (ii), (iii), and (v); and (d) at least two or more of the biomarkers in the biomarker panel comprise the biomarkers in (i), (ii), and (iii).

In a further embodiment, the first analyte-binding ligand or the second analyte-binding ligand comprises an antibody that binds specifically to cotinine or anabasine. In addition, the first analyte-binding ligand or the second analyte-binding ligand further comprises a first soluble capture ligand or a second soluble capture ligand that binds specifically to cotinine or anabasine.

The first soluble capture ligand or the second soluble capture ligand may include a detectable label, e.g., a radioisotope, a fluorescent or chemiluminescent compound, and an enzyme. Examples of detectable label include fluorescein isothiocyanate, rhodamine, luciferin, biotin, alkaline phosphatase, β-galactosidase, and horseradish peroxidase. To measure the detectable label, quantitative immunoassay methods, e.g., competitive binding assay, direct and indirect sandwich assay, immunoprecipitation assay, immunohistochemistry, enzyme-linked immunosorbent assay (ELISA), fluorescence-activated cell sorting (FACS), and Western blot assay are employed. Another approach to quantify the detectable label is to use quantitative methods such as nuclear magnetic resonance (NMR), mass spectrometry (MS), high performance liquid chromatography (HPLC), gas liquid chromatography (GLC) or a combination thereof.

Another embodiment of the invention includes a behavior modification program that can be used for assessing a subject's compliance with a smoking cessation program. The behavior modification program involves the subject being supervised by a health care provider in the smoking cessation program. One or more biological samples from the subject are contacted with a set of biomarkers from the biomarker panel as described hereinabove. The biomarkers are then assayed and compared with reference levels of each corresponding set of reference biomarkers. Based on the comparison results, a determination as to whether the patient is in compliance with the smoking cessation program is performed.

In one embodiment, the behavior modification program further comprises modifying or maintaining the behavior modification program for the subject based on the determination step as described above. The behavior modification program also includes identifying the levels of the set of biomarkers in the biological samples as normal, increased or decreased.

A therapy guidance for the patients can be included with the smoking cessation program. The therapy guidance, according to the embodiment of this invention, involves drug therapy and recommendations on making or maintaining lifestyle choices and reporting to the patient the cardiovascular and/or cancer risk based on the determination outcome. Lifestyle choices may involve changes in diet and nutrition, changes in exercise, smoking elimination and/or a combination thereof. Therapy guidance may further comprise nicotine replacement therapy, drug prescription therapy, nutritional therapy and psychological counseling.

Examples of nicotine replacement therapy may include gums, lozenges and transdermal nicotine-delivery patches. Examples of drugs and nutrition for treating the subject include bupropion (Zyban®), varinicline (Chantix®), a statin drug, niacin, fibrates, dietary supplement with fish oils, cholesterol absorption inhibitors, cholesterol-sequestering resins, Lovastatin/ERN (Advicor®), Simvastatin/ERN (SimCor®), Ezetimibe/Simvastatin (Vytorin®), anti-hypertensive drugs, and blood-glucose-lowering drugs.

A biological sample may include a blood component (serum or plasma), urine or saliva. A biological sample may also include cells in culture, cell supernatants, cell lysates, biological fluids, tissue homogenates and tissue samples.

The behavior modification program may include analyzing a baseline (pre-treatment) sample from the patient and comparing the baseline pretreatment sample to one or more post-treatment samples to assess patient compliance with the smoking cessation program.

The behavior modification program may also include (1) correlating the levels of one or more cardiovascular risk biomarkers from the biomarker panel, as described hereinabove, with the patient's smoking habits with an increased or decreased risk of a cardiovascular disease; (2) correlating the levels of two or more carcinogen biomarkers from the biomarker panel, as described hereinabove, with the patient's smoking habits with an increased or decreased risk of a cancer; and (3) correlating the levels of one or more antioxidants, the levels of one or more oxidative-stress markers; the levels of one or more analytes associated with insulin resistance, glycemia and/or beta cell dysfunction; the levels of the plurality of immuno-markers from an early lung cancer detection test (e.g. EarlyCDT®) and/or the levels of one or more cancer polymorphic genes from the biomarker panel, as described hereinabove, with the patient's smoking habits to determine whether the patient has an increased or decreased risk of a cardiovascular disease or a cancer.

The behavior modification program may further provide positive or negative reinforcement to motivate patient compliance with the prescribed therapy guidance. It is believed that an effective after-patient compliance with the smoking cessation program will decrease the risk of morbidity and mortality by cardiovascular disease and cancer as a result of behavior modifications.

To assess patients' continued compliance and recovery, the assaying, comparing and determining steps in the behavior modification program can be run repeatedly during the course of the smoking cessation program.

Additional aspects, advantages and features of the invention are set forth in this specification, and in part will become apparent to those skilled in the art on examination of the following, or may learned by practice of the invention. The inventions disclosed in this application are not limited to any particular set of or combination of aspects, advantages and features. It is contemplated that various combinations of the stated aspects, advantages and features make up the inventions disclosed in this application.

### DETAILED DESCRIPTION OF THE INVENTION

Due to the aforementioned lack of truthfulness in patients' interactions with their healthcare providers, inaccurate information is likely given to the provider at least for some of the patients. This information may include (1) whether a patient has actually complied and quit smoking; (2) whether a patient has not complied at all with smoking cessation; (3) whether a patient is partially complying by “cutting down”; (4) how often a patient is “cheating” during the smoking cessation program in terms of packs/day; (5) how recently the patient smoked; (6) whether a patient is using nicotine replacement products appropriately; and (7) whether the patient is taking prescription drugs to aid the smoking cessation process, as prescribed. Accurate information on a patient's actual cessation status and compliance is critical to understanding if the patient is in compliance, and if not in compliance, how to change therapies to increase their chance of successfully quitting.

Additionally, because smoking causes biochemical changes that alter the physiology of the smoker, biomarkers for inflammation, oxidative stress, and lipids and lipoproteins associated with high risk of cardiovascular diseases (e.g., stroke, MI, and atherosclerosis) are generally present in smokers at higher levels than in non-smokers. Smoking alters lipid metabolism through increase in lipolysis, insulin resistance and tissue toxicity, leading to elevated levels of plasma lipoproteins. See Amala, G. et al., Impact of Tobacco Smoking on Lipid Metabolism, Body Weight and Cardiometabolic Risk, Current Pharmaceutical Design, 16:2526-02530, 2010, hereby incorporated by reference to its entirety. In addition, a long-term exposure to cigarette smoke causes permanent inflammation and an imbalance in lipid profile, which, in turn, results in an accumulation of lipids in the hepatocytes, leading to non-alcoholic fatty liver disease (NAFLD). See Yuan, H. et al., Second-Hand Smoke Stimulates Lipid Accumulation in the Liver by Modulating AMPK and SREBP-1, J. Hepatol. 51:535-547 (2009), hereby incorporated by reference to its entirety. Smoking incites an immunologic response to vascular injury, described as oxidative stress leading to lipid peroxidation, endothelial cell dysfunction and foam cell proliferation in the tunica media. See Libby, P. et al., Circulation, 105::1135-1143, (2002) and Szmitko, P. E. B. et al., Biomarkers of Vascular Disease Linking Inflammation to Endothelial Activation, Circulation, 108:2041-2048 (2003), both of which are hereby incorporated by reference to their entirety. Furthermore, cigarette smoking is associated with increased levels of inflammatory markers. See Pradhan, A. D. et al., Inflammatory Biomarkers, Hormone Replacement Therapy and Incident Coronary Heart Disease: Perspective Analysis from the Women's Health Initiative Observational Study, JAMA 288:980-987, (2002), hereby incorporated by reference to its entirety. Cigarette smoke also lowers the level of endogenous antioxidants. See El-Zayadi, A. et al., World J, Gastroenterol., 10 2963-2966 (2004), hereby incorporated by reference to its entirety.

Generally speaking, it is expected that if a patient successfully quits smoking, many biomarkers of cardiovascular risk will normalize with time and the patient's risk of morbidity and mortality from cardiovascular disease will decline. However, due to other disease processes of the body and/or genetic predispositions to diseases, and/or patient lifestyles, the cardiovascular risk may not decline as measured by some analytes. This presents another challenge in patient management for health-care providers. Diagnostic testing to correlate smoking cessation compliance with cardiovascular risk biomarkers allows the healthcare provider to (1) determine with greater accuracy the patient's actual tobacco-use status and (2) distinguish inherent cardiovascular disease risk from disease risk brought on by smoking. Without testing both for compliance and cardiovascular risk together, an incomplete and inaccurate assessment could result in sub-optimal therapy and increased likelihood of adverse events, including morbidity and mortality resulting from cardiovascular disease.

Cigarette smoke is a complex aerosol that contains a deadly mix of more than 7,000 chemicals; hundreds are toxic and about 70 can cause cancer. It also causes biochemical changes to the body that increase the risk of developing cancer. This is partially due to the inflammatory properties of the chemicals in tobacco smoke and the accumulation of carcinogens which can directly promote DNA damage, mutations, and growth of tumors. Many of these carcinogens have been identified and are present in blood and urine, and are known to decline as they are metabolized and cleared from the body at varying rates with smoking cessation. It is therefore possible to measure a panel of carcinogens to determine whether someone is actively smoking or being exposed to passive “second hand” smoke. Also, because some carcinogens have much longer half-lives than others, it is possible to determine how long a given patient has been smoke-free. Lastly, because most of these carcinogens are derived from the burning of tobacco, it is possible to measure declines of these chemicals in the patient who is quitting by means of nicotine replacement therapy.

Besides external factors like carcinogen exposure, internal factors like patient genetic polymorphisms at some alleles make patients much more likely to develop cancer from smoking and other tobacco-related uses. Thus, measuring the levels of tobaccco carcinogens and screening for genetic polymorphisms associated with cancer due to smoking may be a useful tool for risk management during the smoking cessation process by the healthcare provider as well as for motivating patients to comply with a smoking cessation program.

The biomarker panels and behavioral modification programs are employed for assessing a subject's compliance with a smoking cessation program or regimen based on the detection of non-compliance in form of continued smoking, use of nicotine replacement therapies, use of drug therapies, and the impact of the therapy compliance on a subject's attendant risk of morbidity and mortality from cardiovascular diseases and cancer.

The advantages and uniqueness of employing the biomarker panels and behavioral programs are based on the combination of assessing patient compliance in the setting of smoking cessation along with assessing changes in both the cardiovascular and cancer risks of subjects, and the use of the information by a healthcare provider as a tool to assess changes in risk of cardiovascular disease and cancer, effect therapy to minimize these risks, and change patient behavior by motivating compliance.

### Nicotine and Nicotine Metabolites (Cotinine and Anabasine)

“Nicotine metabolite” refers to a product of metabolism of nicotine by a body tissue and/or body fluid. Nicotine metabolites include, but are not limited to cotinine (COT), 3-hydroxycotinine (3HC), 5-hydroxycotinine (5HC), cotinine-glucuronide (COT-glucuronide), 3-hydroxycotinine-glucuronide (3HC-glucuronide), cotinine-N-oxide (COT-N-oxide) (CNO), Norcotinine, Nornicotine, Nicotine-N-oxide (NNO), and Nicotine-glucuronide.

“Cotinine” and “COT” are used interchangeably.

“Tobacco alkaloid” refers to chemical compounds that exist naturally in tobacco. These compounds are transferred to smokers or smokeless tobacco users through contact with tobacco and are not usually products of metabolism, however, some tobacco alkaloids are also nicotine metabolites which occur both naturally in tobacco and are synthesized through metabolism of nicotine. Nicotine is an example of a tobacco alkaloid while Nornicotine is both a tobacco alkaloid and a nicotine metabolite. Tobacco alkaloids include, but are not limited to Nicotine, Nornicotine, Anabaseine, Anatabine, Nicotelline, and Nicotyrine.

During the last forty years, there has been an increasing focus on cigarette smoking and the adverse health consequences associated with it. Most people are aware of the dangers of smoking and many are aware that nicotine causes addiction and that it may play a role in the development of cardiovascular disease and reproductive disturbances. See Benowitz, N. L., Smoking-Induced Coronary Vasoconstriction: Implications for Therapeutic Use of Nicotine, J. Am. Coll. Cardiol. 22:648-649 (1993) and Benowitz, N. L., Pharmacology of Nicotine: Addiction and Therapeutics, Annu Rev. Pharmacol. Toxicol. 36:597-613 (1996), both of which are hereby incorporated by reference to their entirety. Nicotine absorbed onto pulmonary circulation distributes rapidly to brain and heart tissues. It reaches the central nervous system within 20 s of tobacco smoke inhalation and has a short distribution (8 min) and elimination (2 hr) half-lives. The liver is the primary site of nicotine metabolism, although the lung and kidney also contribute to metabolism. A small fraction of nicotine is metabolized to nornicotine. 70% of circulating nicotine is metabolized to cotinine by cytochrome P450 and aldehyde oxidase. Unlike nicotine, cotinine has a longer half-life of about 20 hrs. See Benowitz, N. L., The Human Pharmacology of Nicotine, Res. Adv. Alcohol Drug Probl. 9:1-52 (1986), hereby incorporated by reference to its entirety. Cotinine is further metabolized to trans-3′ hydroxycotinine. On average, 30% of the nicotine that enters the liver exits unchanged, and 70% leaves the liver in metabolite forms. Cotinine undergoes further metabolism to trans-3′-hydroxycotinine.

Quantification of nicotine and its metabolites in the biological samples is very beneficial not only to tobacco users whose desire is to quit smoking but also to the physicians and healthcare providers whose main goal is to identify the patients (1) who are still smoking; (2) who have stopped smoking; and (3) who showed a relapse from smoking If results of the tests indicate that the patient is actively using a tobacco product during therapy, additional counseling or intervention may be proper. Nicotine can be found in urine samples of tobacco users at a concentration range of about 1,000-5,000 ng/mL. Nicotine is rapidly metabolized to cotinine and trans-3-hydroxycotinine and has a half life of 2 hours. In contrast, cotinine and trans-3-hydroxycotinine are excreted in the urine of tobacco users in the range of 1,000-8,000 ng/mL and 3,000-25,000 ng/mL, respectively. The half-lives of cotinine and trans-3-hydroxycotinine are 15 and 10 hours, respectively. High-dose nicotine patch therapy can also produce similar concentrations of nicotine. Urine nicotine, cotinine and trans-3-hydroxycotinine in tobacco users at the above-mentioned ranges indicate that the tobacco users are either actively using a tobacco product or on high-dose nicotine patch therapy. Nicotine and cotinine cannot, however be used to assess tobacco use in subjects using nicotine gum, transdermal patches, nicotine inhalers or other nicotine medications. Since minor tobacco alkaloids such as anabasine, nornicotine and anatabine are present in tobacco but absent in nicotine-containing medications, their measurements may be useful for detecting tobacco use in persons undergoing nicotine replacement therapy. See Peyton, J. III, et al., Minor Tobacco Alkaloids as Biomarkers for Tobacco Use: Comparison of Users of Cigarettes, Smokeless Tobacco, Cigars, and Pipes, Am. J. Public Health, 89:731-736 (1999), hereby incorporated by reference to its entirety. Accordingly, these tobacco alkaloids may also serve as unique markers of tobacco use. Urinary anabasine and nornicotine have a concentration range of at least about >10 ng/mL and >30 ng/mL, respectively, regardless of whether the tobacco user is undergoing nicotine replacement therapy. See Moyer, T. P. et al., Simultaneous analysis of nicotine, nicotine metabolites and tobacco alkaloids in serum or urine by tandem mass spectrometry, with clinically relevant metabolic profiles, Clin. Chem., 48:1460-1471 (2002), hereby incorporated by reference to its entirety. Table 1 provides the possible ranges of nicotine, cotinine, trans-3-hydroxycotinine, anabasine and nornicotine for individuals who are still using a tobacco product, individuals who have completely abstained from tobacco use for 2-weeks, nontobacco user with passive exposure (individuals involuntarily exposed to environmental or second-hand tobacco smoke) to tobacco smoke and nontobacco user with no passive exposure. Passive exposure to tobacco can cause the accumulation of nicotine metabolites among nontobacco users. Nicotine and its metabolites may be extracted from urine using solid-phase extraction techniques. The extract eluate can be further quantified by high-performance liquid chromatography-tandem mass spectrometry. See Moyer, T. P. et al., Simultaneous analysis of nicotine, nicotine metabolites and tobacco alkaloids in serum or urine by tandem mass spectrometry, with clinically relevant metabolic profiles, Clin. Chem., 48:1460-1471 (2002), hereby incorporated by reference to its entirety. Xu, X, et al. also reported of a highly sensitive LC/MS/MS method for simultaneously detecting nicotine and its metabolites, as well as anabasine, across a wide range of concentrations in urine specimens after a simple solid-phase extraction. See Xu, X. et al., Clin. Chem., 50(12):2323-2330 (2004), hereby incorporated by reference to its entirety.

TABLE 1

| | Active tobacco | | | |
|---|---|---|---|---|
| | users or | Tobacco | Non-tobacco | Non-tobacco |
| | high dose | user after | user with | user with |
| | nicotine patch | 2 weeks' | Passive | no passive |
| Concentration | therapy | abstinence | exposure | exposure |
| of Urinary: | (ng/mL) | (ng/mL) | (ng/mL) | (ng/mL) |
| Nicotine | 1,000-5,000 | <30 | <20 | <2.0 |
| Cotinine | 1,000-8,000 | <50 | <20 | <5.0 |
| trans-3-OH- | 3,000-25,000 | <120 | <50 | <50 |
| cotinine | | | | |
| Anabasine | 3-500 | <2.0 | <2.0 | <2.0 |
| Nornicotine | 30-900 | <2.0 | <2.0 | <2.0 |

Adapted from Moyer, T. P. et al., Clin. Chem. 48(9): 1460-1471(2002)

Cotinine ELISA test kits for urine, serum, plasma samples are commercially available. An example of a commercially available cotinine ELISA test kit for urine test that employs a monoclonal anti-cotinine antibody is Status DS™ Nicotine Test Kit by Princeton BioMeditech (PBM) Corporation (Monmouth Junction, N.J.; see Pojer R. et al., Clin Chem. 30(8):1377-1380 (1984), hereby incorporated by reference to its entirety). NicAlert™ Cotinine Test (manufactured by Nymox Pharmaceutical Corporation, Hasbrouck Heights, N.J.), is available to measure cotinine in saliva samples (including urine). The saliva test kit uses a cotinine-specific monoclonal antibody attached to gold particles to measure cotinine in a semi-quantitative and immunochromatographic strip (“dipstick” format). The saliva NicAlert™ cotinine test kit has a sensitivity of 99% and a specificity of 96%. See Montalto, N. J. and Wells, W. O., Validation of Self-Reported Smoking Status Using Saliva Cotinine A Rapid Semiquantitative Dipstick Method, Cancer Epidemiol. Biomarkers Prev., 16(9):1858-1862 (2007), hereby incorporated by reference to its entirety.

Anti-cotinine antibodies have been developed and generated, examples of which include (1) a rabbit/human chimeric monoclonal antibody specific to cotinine that contains rabbit heavy chain (VH) and light chain (VL) variable domain and human heavy chain (CH1) and light chain (CL) constant domain (Park, S. et al., U.S. Pat. No. 8,008,448, hereby incorporated by reference to its entirety); (2) a mouse monoclonal antibody to cotinine (prepared by first covalently coupling a cotinine conjugate to keyhole lympet hemocyanin (KLH)) to produce cotinine-KLH conjugate/immunogen and immunizing the mice with the cotinine-KLH conjugate/immunogen (Langone, J. J. et al., EP 0194158 A2 and Bjercke, R. J. et al., J. Immunol. Methods, 90(2):203-213 (1986), both of which are hereby incorporated by reference to their entirety); and (3) cotinine-conjugated aptamer/anti-cotinine antibody complexes (see Park, S. et al., Exp. Mol. Med. 44(9):554-561 (2012), hereby incorporated by reference to its entirety).

Methods for measuring the level of cotinine and/or anabasine in a sample, such as serum, urine or saliva sample, are well known in the art. Table 2 provides a list of various quantitative methods developed by researchers for measuring cotinine and/or anabasine that are applicable to the embodiments of the invention.

TABLE 2

| | Biological | Quantitative | |
|---|---|---|---|
| Metabolite(s) | Samples | Methods | References* |
| Cotinine | plasma, | LC/MS-MS (SPE) | Moyer, T. P. et al., Clin. Chem. 48(9): 1460-1471 (2002) |
| | serum, | | |
| | urine | | |
| Cotinine | saliva | LC/MS-MS (SPE) | Shakleya, D. M. et al., Anal. Bioanal. Chem. 395(7): 2349-2357 (2009) |
| Cotinine | plasma | LC/MS-MS (SPE) | Shakleya, D. M. et al., J. Chromatogr. B. Analyt. Technol. Biomed. Life |
| | | | Sci. 877(29): 3537-3542 (2009) |
| Cotinine | urine | HPLC-UV (LLE) | Rabbaa-Khabbaz, L., et al., J. Chromatogr. Sci. 44: 535-538 (2006) |
| Cotinine | plasma, | LC-MS/MS (ESI) | Jacob III, P. et al., J. Chromatogr. B. Analyt. Technol. Biomed. Life |
| | urine, | LC-MS/MS (APCI) | Sci. 879(3-4): 267-276 (2011) |
| | saliva | | |
| cotinine, | urine | LC/MS-MS (SPE) | Xu, X. et al., Clin. Chem. 50(12): 2323-2330 (2004) |
| anabasine | | | |
| cotinine, | urine | LC/MS-MS (CC) | Hoofnagle, A. N. et al., Am. J. Clin. Pathol.126(6): 880-887 (2006) |
| anabasine | | | |
| cotinine, | urine, | LC-MS (SPME) | Kataoka, H. et al., J. Pharm. Biomed. Anal. 49(1): 108-114 (2009) |
| anabasine | saliva | | |
| cotinine, | plasma, | LC-MS/MS (ESI) | Miller, E. I. et al., J. Chromatogr. B. Analyt. Technol. Biomed. Life Sci. |
| anabasine | urine | | 878(9-10): 725-737 (2010) |
| cotinine, | plasma, | LC/MS-MS (SPE) & | Miller, E. I. et al., J. Anal. Toxicol. 34: 357-366 (2010); Cotinine ELISA |
| anabasine | urine | ELISA | Kit from Immunalysis. |
| cotinine, | urine, | GC-FID (SDME) | Kardani, F. et al., J. Chromatogr. B. Analyt. Technol. Biomed. Life Sci. |
| anabasine | saliva | | 878(28): 2857-62 (2010) |

Abbreviations:
LC/MS-MS—liquid chromatography/tandem mass spectrometry;
SPE—solid phase extraction;
LLE—liquid-liquid extraction;
ESI—electrospray ionization;
APCI—atmospheric pressure chemical ionization;
CC—centrifugal clarification;
SPME—solid phase microextraction;
GC-FID—gas chromatography-flame ionization detection;
SDME—single-drop microextraction.
*The references cited hereinabove are hereby incorporated by reference to their entirety.

Besides employing the methods provided in Table 2 to measure cotinine and anabasine, an immunodetection method such as ELISA (Enzyme-Linked-Immunosorbent Assay) can be used to measure or quantitate the level of cotinine in a biological sample of a subject undergoing the smoking cessation program or receiving therapeutic intervention and health risk management due to smoking. An agent that specifically binds anti-cotinine antibody can be used with the ELISA method.

Immunogens useful for producing antibodies to cotinine contain a hapten conjugated to an immunogenic carrier. Any immunogenic carrier effective to cause an immune response to the immunogen may be bound to the hapten. In some embodiments, the immunogenic carrier is a large molecule, such as a protein, having the ability to provoke an immune response when administered to an animal. Suitable immunogenic carriers include proteins, polypeptides or peptides such as albumins, hemocyanins, thyroglobulins and derivatives thereof, particularly bovine serum albumin (BSA) and keyhole limpet hemocyanin (KLH), polysaccharides, carbohydrates, polymers, and solid phases. Other protein derived or non-protein derived substances are known to those skilled in the art. An immunogenic carrier typically has a molecular weight of at least 1,000 daltons, preferably greater than 10,000 daltons. Carrier molecules often contain a reactive group to facilitate covalent conjugation to the hapten. The carboxylic acid group or amine group of amino acids or the sugar groups of glycoproteins are examples of groups often used in this manner. Carriers lacking such groups are often reacted with an appropriate chemical to produce them. Preferably, an immune response is produced when the immunogen is administered to animals such as mice, rabbits, rats, goats, sheep, guinea pigs, chickens, and other animals, most preferably by the injection mice or rabbits.

### Tobacco Carcinogen Biomarkers

Examples of tobacco carcinogen biomarkers include tobacco carcinogens or their metabolites in urine, breath, blood nails and hair and other biological samples; tobacco carcinogen-DNA adducts; and tobacco carcinogen-protein adducts.

Tobacco metabolite biomarkers may include metabolites of polycyclic aromatic hydrocarbons (PAH) and tobacco-specific nitrosamines and mercapturic acids derived from benzene, acrolein, and 1,3-butadiene. An example of a PAH metabolite is 1-hydroxypyrene (1-HOP; a pyrene metabolite), a non-carcinogen but a component of all PAH mixtures. IHOP can be measured by HPLC with fluorescence detection and has been used to assess PAH uptake. IHOP level has been reported to be 2-3 times higher in smokers than nonsmokers. See Hecht, S. S., Tobacco Smoke Carcinogens and Lung Cancer, J. Natl. Cancer Inst. 91(14):1194-1210 (1999), hereby incorporated by reference to its entirety. Another metabolite biomarker is r-1,t-2,3-c-4-tetrahydrophenanthrene (PheT) which maybe applied as a PAH uptake and metabolic activation marker. PheT is found abundantly in urine, which facilitates its measurement. See Hecht, S. S. et al., r-1,t-2,3,c-4-Tetrahydroxy-1,2,3,4-tetrahydrophenanthrene in Human Urine: A Potential Biomarker for Assessing Polycyclic Aromatic Hydrocarbon Metabolic Activation, Cancer Epidemiol. Biomarkers Prev., 12(12):1501-1508 (2003) and Yuan, J. M. et al., Urinary Levels of Cigarette Smoke Constituent Metabolites are Prospectively Associated with Lung Cancer Development in Smokers, Cancer Res., 71(21):6749-6757 (2011), both of which are hereby incorporated by reference to their entirety.

Additional tobacco carcinogen biomarkers include the metabolites of mercapturic acids derived from benzene, acrolein and 1,3-butadiene. They include but are not limited to: 1-hydroxy-2-(N-acetylcysteinyl)-3-butene and 1-(N-acetylcysteinyl)-2-hydroxy-3-butene [collectively called MHBMA for monohydroxybutylmercapturic acid]; 1,2-dihydroxy-4-(N-acetylcysteinyl)butane [DHBMA for dihydroxybutylmercapturic acid], metabolites of 1,3-butadiene; 1-(N-acetylcysteinyl)-propan-3-ol (HPMA for 3-hydroxypropyl mercapturic acid), a metabolite of acrolein; 2-(N-acetylcysteinyl)butan-4-ol (HBMA for 4-hydroxybut-2-yl mercapturic acid), a metabolite of crotonaldehyde; (N-acetylcysteinyl)benzene (SPMA for S-phenyl mercapturic acid), a metabolite of benzene; (N-acetylcysteinyl)ethanol (HEMA for 2-hydroxyethyl mercapturic acid), a metabolite of ethylene oxide; 1-hydroxypyrene and its glucuronides (1-HOP), metabolites of pyrene. See Carmella, S. G. et al., Effects of Tobacco Cessation on Eight Urinary Tobacco Carcinogen and Toxicant Biomarkers, Chem. Res. Toxicol., 22(4):734-741 (2009), hereby incorporated by reference to its entirety.

Cigarette carcinogens such as benzene, acrolein, crotonaldehyde, benzene oxide and 1,3-butadiene are found in the gas phase of the cigarette smoke while PAH and NNK, represented by IHOP and total NNAL are particulate phase constituents. With the exception of DHBMA, levels of which did not change after cessation of smoking, all other tobacco carcinogen biomarkers decreased significantly after three days of cessation (P<0.001). The decreases in MHBMA, HPMA, HBMA, SPMA, HEMA, I—HOP, and NNK are related to smoking and are good indicators on the impact of smoking on human exposure to benzene, acrolein, crotonaldehyde, benzene oxide, 1,3-butadiene, PAH and NNK. See Carmella, S. G. et al., Effects of Tobacco Cessation on Eight Urinary Tobacco Carcinogen and Toxicant Biomarkers, hereby incorporated by reference to its entirety.

Various methods including gas chromatography-mass spectrometry (GC-MS), gas chromatography-tandem mass spectrometry (GC-MS/MS and LC-MS/MS may be used to quantitate and measure MHBMA and DHBMA. HPMA, HBMA, SPMA and HEMA can all be analyzed by LC-MS/MS.

Metabolites of nicotine (cotinine, trans-3′-hydrozycotinine and their glucoronides) and NNK (NNAL (4-(methylnitrosamino)-1-(3-pyridyl)-1-butanol) and its glucoronides) can be measured in secondhand smoke exposed (SHSe) individuals with high sensitivity in various biological samples such as urine, blood, saliva, toenails, and hair. Because of its sensitivity and specificity, NNAL is the only tobacco-specific biomarker that is consistently elevated in non-smokers exposed to second hand cigarette smoke. See Tobacco, Science, Policy and Public Health, 2nd Edition, Chapter 7, pp. 127-154, Boyle, P. et al. (eds), Oxford University Press, Inc., Oxford, N.Y., 2010, hereby incorporated by reference to its entirety. Nicotine is extensively metabolized in the liver and cotinine is its major proximate metabolite. Cotinine has a longer half-life (average t½=16 h) than nicotine (t½=2 h) and its concentrations are more stable throughout the day making it a preferred blood, saliva and urine biomarker for SHSe. Cotinine can be measured radioimmunoassay (RIA), gas chromatography-nitrogen phosphorus detection (GC-NPD), gas chromatography-mass spectrometry (GC-MS), liquid chromatography-atmospheric pressure chemical ionization tandem mass spectrometry (LC-APCI MS/MS) and high performance liquid chromatography (HPLC). Nicotine can be measured by GC-MS and HPLC. See Avila-Tang, E. et al., Assessing Secondhand Smoke Using Biological Markers, Tobacco Control, 1-8 (2012), hereby incorporated by reference to its entirety.

The tobacco-specific NNK (4-(N-nitrosomethylamino)-1-(3-pyridyl)-1-butanone) is a nitrosamine and highly effective lung carcinogen. NNK is an important contributor to the etiology of lung and pancreatic cancer in smokers, oral cancer in smokeless tobacco users and lung cancer in people exposed to environmental tobacco smoke. NNK is metabolized in the body to NNAL and NNAL glucoronides (NNAL-gluc, the sum of NNAL-O-Gluc and NNAL-N-Gluc), which are both commonly measured together as total NNAL. See Hecht, S. S., Human Urinary Carcinogen Metabolites: Biomarkers for Investigating Tobacco and Cancer. Carcinogenesis (Lond.), 23:907-922 (2002), hereby incorporated by reference to its entirety. Treatment of urine with β-glucoronidase converts NNAL-O-Gluc and NNAL-N-Gluc. Total NNAL remains in the body longer as cotinine (t½=10 days to 3 weeks) than cotinine. NNAL and its glucoronides (NNAL-Gluc) measurements are absolutely tobacco-specific, directly relevant to carcinogen uptake and consistently detectable in exposed individuals. The assay of choice for NNAL is LC-MS/MS with a sensitivity range of 0.25 pg/ml. A more difficult but more sensitive method for measuring NNAL is by gas chromatography-thermal energy analysis (GC-TEA) with a sensitivity range of 0.15 pg/ml. See Avila-Tang, E. et al. supra and Carmella, S. G. et al., Analysis of Total 4-(Methylnitrosamino)-1-(3-Pyridyl)-1-Butanol (NNAL) in Human Urine, Cancer Epidemiol. Biomarkers Prev., 12:1257-1261 (2003), hereby incorporated by reference to its entirety.

### Tobacco DNA and Protein Adducts

Many of the tobacco carcinogens would require metabolic activation for DNA binding but some can form adducts without metabolism. Examples of tobacco smoke constituents that form DNA adducts include BaP, NDMA, acrolein, ethylene oxide, acetaldehyde, and 4-aminobiphenyl. For general review on tobacco carcinogen-DNA adducts; and tobacco carcinogen-protein adducts, see Hecht, S. S., Tobacco Carcinogenesis: Mechanisms and Biomarkers, Chapter 7, pp. 127-154, in Tobacco Science, Policy and Public Health, 2nd Edition, Boyle, P. et al. (eds), Oxford University Press, Inc., Oxford, N.Y., 2010, hereby incorporated by reference to its entirety.

The formation of BaP-DNA adducts involves conversion of BaP to mutagenenic “bay region” diol epoxides. Competing with BaP metabolic activations are detoxification reactions to produce phenols, dihydrodiols, and glutathione, glucuronide and sulphate conjugates. Quinone metabolites are also commonly observed and they result from initial 6-hydroxylation followed by further oxidation. For NNK and its metabolite, 4-(methylnitrosamino)-1-(3-Pyridyl)-1-Butanol (NNAL), the major metabolic activation pathways are the hydroxylation of the carbons next to the N-nitroso group (α-hydroxylation) leading, via diazonium salts, to DNA adducts. Glucuronidation of NNAL, at either the hydroxyl group or the nitrogen atom of the pyridine ring and pyridine-N-oxidation of NNK and NNAL are detoxification pathways. Metabolic activation of NDMA by α-hydroxylation leads to formation of unstable α-hydroxylNDMA, which can spontaneously lose formaldehyde and produce diazonium ions that react with DNA to form DNA adducts such as 7-methyldeoxyguanosine (7-methyl-dG) and O6-methyl-dG. Denitrosation, which produces nitrite and methylamine, is a detoxification pathway. α-hydroxylation of NNN produces reactive diazonium ions and consequent DNA adducts. β-hydroxylation of NNN and pyridine-N-oxidation are detoxification reactions. NNN is also detoxified by denitrosation/oxidation yielding norcotinine and by glucoronidation of the pyridine ring. Acrolein, ethylene oxide and acetaldehyde directly react with DNA to form well-characterized adducts. There are competing metabolic detoxification pathways involving glutathione conjugation and in the case of acetaldehyde, oxidation. 4-aminobiphenyl is metabolically activated to reactive electrophiles by initial N-hydroxylation. Conjugation of resulting hydroxylamine with acetate or other groups such as sulphate ultimately results in the production of nitrenium ions which react with DNA to form adducts mainly at C-8 of dG. Other aromatic amines and heterocyclic aromatic amines are metabolically activated in similar ways. Acetylation of 4-aminobiphenyl can be a detoxification pathway if it is not followed by N-hydroxylation. Ring hydroxylation and conjugation of the phenolic metabolites result in detoxification.

The major DNA adduct formed from BaP (and several other PAH) results from trans-addition of BPDE to the N2 position of dG. Pyridyloxobutyl (POB)-DNA adducts of NNK and NNN are formed at the 7- and 06-positions of dG, the O2-position of thymidine, and the O2 position of deoxycytidine. Metabolic activation of NNK also results in the formation of 7-methyl-dG and O6-methyl-dG, identical to the DNA adducts formed from NDMA (and other DNA methylating agents). Ethylating agents and ethylene oxide in cigarette smoke also alkylate dG. Acrolein and its homologue, crotonaldehyde react with DNA to produce exocyclic 1,N2-dG adducts while acetaldehyde forms a Schiff base adduct with exocyclic (N2) amino group of dG.

In sum, reactive intermediates from the tobacco carcinogens that are critical in forming DNA adducts include diol epoxides of PAH, diazonium ion products of nitrosamine α-hydroxylation and nitrenium ions produced from esters of N-hydroxylated aromatic amines. Some other cigarette smoke compounds such as ethylene oxide, acrolein and acetaldehyde react directly with DNA to form adducts. Glutathione and glucuronide conjugation are particularly important in the detoxification of cigarette smoke carcinogens.

Cigarette smoke causes oxidative damage probably because it contains free radicals such as nitric oxide, as well as mixture of catechols, hydroxyquinones, semiquinones and quinones, which can reduce redox recycling. Smokers have lower level of ascorbic acid, higher levels of oxidized lipids and sometimes higher levels of oxidized DNA such as 8-oxo-dG than non-smokers. See Hecht, S. S., Tobacco Smoke Carcinogens and Lung Cancer, J. Natl. Cancer Inst. 91(14):1194-1210 (1999), hereby incorporated by reference to its entirety.

Carcinogen DNA adducts are challenging to quantify because of their extremely low levels and low tissue or blood sample availability. However, measurements have improved using mass spectrometers such as LC-MS/MS. Quantitative methods such as HPLC-fluorescence, HPLC with electrochemical detection, and post-labelling and immunoassay techniques can also be used. Besides blood samples and lung tissues, adduct levels can also be detected in larynx, oral and nasal mucosa, bladder, cervix, breast, pancreas, stomach, placenta, fetal tissue, cardiovascular tissues, sputum and sperm. See Phillips, D. H., Smoking-Related DNA and Protein Adducts in Human Tissues, Carcinogenesis, 23(12):1979-2004 (2002) and Hecht, S. S., Tobacco Carcinogenesis: Mechanisms and Biomarkers, Chapter 7, pp. 127-154, in Tobacco Science, Policy and Public Health, 2nd Edition, Boyle, P. et al. (eds), Oxford University Press, Inc., Oxford, N.Y., 2010, both of which are hereby incorporated by reference to their entirety.

### Carcinogen-Protein Adducts

Carcinogen-protein adducts have been used as surrogates for DNA adduct measurements. The protein-adducts can be measured by mass spectrometry to detect the carcinogen moiety after its release from protein by mild acid or base hydrolysis or release and detection of the modified N-terminal valine of haemoglobin; immunochemical analysis using antibodies raised against protein adducts; and HPLC with fluorescence detection of the released carcinogen. Haemoglobin and albumin adducts have been extensively studied as biomarkers of human exposure to environmental carcinogens. See Phillips, D. H., Smoking-Related DNA and Protein Adducts in Human Tissues, Carcinogenesis, 23(12):1979-2004 (2002), hereby incorporated by reference to its entirety. Examples of carcinogen protein adducts include but are not limited to adducts formed by aromatic amines (e.g., 4-ABP, 3-ABP, 2-naphthylamine, o-toluidine, p-toluidine, 2-ethylaniline and 2,4-dimethylaniline adducts); polycyclic aromatic hydrocarbons (PAH; e.g., BPDE, chrysene); tobacco-specific nitrosamines (e.g., 4-hydroxy-1-(3-pyridyl)-1-butanone (HPM) derived from NNK or NNN); ethylene (ethylene converts the N-terminal valine of haemoglobin, via ethylene oxide, to N-(2-hydroxyethyl)valine (HOEtVal)); acrylamide; acrylotnitrile (N-(2-cyanoethyl)valine); and benzene (e.g., benzene oxide or 1,4-benzoquinone). See Phillips, D. H., Smoking-Related DNA and Protein Adducts in Human Tissues, Carcinogenesis, 23(12):1979-2004 (2002), hereby incorporated by reference to its entirety.

Advantages of Hb adducts as surrogates include the ready availability of relatively large amounts of Hb and the relatively long lifetime of the erythrocyte in humans (120 days). Hemoglobin adducts of aromatic amines such as 3-aminobiphenyl- and 4-aminobiphenyl-Hb adducts are consistently higher in smokers than in non-smokers.

### Anti-Oxidants

An “antioxidant,” as used herein, refers to a substance that inhibits oxidation or reactions promoted by oxygen or peroxides. Anti-oxidants offer some protection against tobacco-related diseases, e.g., lung cancer and esophageal cancer. Surveys conducted in the United States and several European universities revealed that smokers with diets deficient in antioxidants had twice as much lung cancer as smokers whose antioxidants and vitamins intake was not deficient. At first, these patients were treated with high does of conventional anti-cancer drugs, but they suffered from side effects of this therapy before any effect on their cancer could be documented. When researchers begun experimenting with β-carotene and retinoic acid derivatives, a sensible improvement was immediately noted. See U.S. Patent Application No. 20050241658, hereby incorporated by reference to its entirety. In addition, anti-oxidants may be beneficial to smokers who are facing stressful conditions and/or experiencing unpleasant sensations like irritability, drowsiness, anxiety, headaches, difficulty to concentrate, which normally resulting from total nicotine withdrawal. Non-limiting examples of antioxidants that are usable according to the embodiment of the invention include, but not limited to, vitamin A and its analogs and derivatives, ascorbic acid (vitamin C and its salts, ascorbyl esters of fatty acids, ascorbic acid derivatives, tocopherol (vitamin E) tocopherol sorbate, tocopherol acetate, other tocopherol esters, vitamin B₂, B₆ and B₁₂ compounds, sulfhydryl compounds (e.g. L-glutathione), lutein, lycopene and β-carotene. It will be understood in this art that various other anti-oxidants not included in the above-mentioned non-limiting examples can also be used.

### Genetic Polymorphisms

Tobacco smoke contains an array of potent chemical carcinogens and reactive oxygen species that may produce DNA bulky adducts, crosslinks, oxidative or base DNA damage and DNA strand breaks. Among the major DNA repair pathways that operate on specific types of damaged DNA by cigarette smoking, base-excision repair (BER) is involved in the repair of DNA base damage and single strand breaks and, on the other hand, nucleotide excision repair (NER) participates in the repair of bulky monoadducts, cross links and oxidative damages. For double strand breaks, the DNA double strand break (DSB) pathway is involved.

Polymorphisms in genes involved in the metabolic activation (activating Phase 1 enzymes; cytochrome P450) and detoxification (detoxifying Phase II enzymes; glutathione S-transferase) of tobacco carcinogens as well as in the repair of DNA damage (8-oxoguanine DNA glycosylase 1) have all been associated with an increased of lung cancer in case-control studies. These polymorphic genes may include, but are not limited to, myeloperoxidase (MPO); cytochrome P450 CYP1A1 Ile462Val and CYP1B1 Leu462Val; glutathione S-transferase subclass of enzymes, GSTM1 (mu), GSTT1 (theta) and GSTP1 (pi); x-ray repair cross-complementing group 1 (XRCC1; Arg399Gln)); x-ray repair cross-complementing group 3 (XRCC3; Thr241Val)); excision repair cross-complementing group 1 (ERCC1; Asn118Asn); excision repair cross-complementing group 1 (ERCC2 or also known as xeroderma pigmentosum group d protein (XPD) XPD (Lys751Gln, Asp312Asn)); 8-oxoguanine DNA glycosylase (OGG1 (Ser326Cys)); and SUV39H2 histone methyltransferase. See Larsen, J. E. et al., CYP1A1 Ile462Val and MPO G-463A Interact to Increase Risk of Adenocarcinoma But Not Squamous Cell Carcinoma of the Lung, Carcinogenesis, 27:525-532 (2006); Wenzlaff, A. S. et al., CYP1A1 and CYP1B1 polymorphisms and Risk of Lung Cancer Among Never Smokers: a Population-Based Study. Carcinogenesis, 26:2207-2212 (2005); Wenzlaff, A. S. et al., GSTM1, GSTT1 and GSTP1 Polymorphisms, Environmental Tobacco Smoke Exposure and Risk of Lung Cancer Among Never Smokers: a Population-Based Study. Carcinogenesis, 26:395-401 (2005); Le Marchand, L. et al., Association of the hOGG1 Ser326Cys Polymorphism with Lung Cancer Risk, Cancer Epidemiol. Biomarkers Prev., 11:409-412 (2002); Yin, Z. et al., Association Between Polymorphisms in DNA Repair Genes and Survival of Non-Smoking Female Patients with Lung Adenocarcinoma, BMC Cancer 9:439455 (2009); Hodgson, M. E. et al., Smoking and Selected DNA Repair Gene Polymorphisms in Controls: Systematic Review and Analysis, Cancer Epidemiol. Biomarkers Prev., Vol. 19(12):3055-3086 (2010); Hoffmann, H. et al., Genetic Polymorphisms and the Effect of Cigarette Smoking in the Comet Assay, Mutagenesis, 20(5):359-364 (2005); Yin, Z. et al., Association Between Polymorphisms in DNA Repair Genes and Survival of Non-Smoking Female Patients with Lung Adenocarcinoma, BMC Cancer, 9:439-445 (2009); Zhou, W. et al., Polymorphisms in the DNA Repair Genes XRCC! And ERCC2, Smoking and Lung Cancer Risk, Cancer Epidemiol. Biomarkers Prev., 23:359-365 (2003), all of which are hereby incorporated by reference to their entirety.

Human 8-Oxoguanine DNA Glycosylase (hOGG1)

The human OGG1 gene encodes a DNA glycosylase/AP-lyase that catalyzes the removal of 8-hydroxy-2′-deoxyguanine (8-OH-dG) adducts as part of the base excision repair (BER) pathway. The hOGG1 gene is located on chromosome 3p26.2, a region that shows frequent loss of heterozygosity in several human cancers. See Shinmura, K. et al., The OGG1 Gene Encodes a Repair Enzyme for Oxidatively Damaged DNA and Is Involved in Human Carcinogenesis, AntiOxid. Redox Signal, 3:597-609 (2001), hereby incorporated by reference to its entirety. A C→G sequence variant leading to an amino acid change from serine to cysteine at codon 326 (Ser326Cys) has been suggested to might be associated with increase risk for lung and esophageal cancers. See Elahi, A. et al., The Human OGG1 DNA Repair Enzyme and Its Association With Otolaryngeal Cancer Risk, Carcinogenesis, 23(7):1229-1234 (2002), hereby incorporated by reference to its entirety. Benzo[a]pyrene was shown to induce 8-OH-dG and a link between 8-OH-dG formation and tobacco smoke carcinogenesis based on the elevated levels of 8-OH-dG were observed in lung DNA of smokers (as compared with non-smokers), with a correlation observed between levels of 8-OH-dG and the number of cigarettes smoked. See Asami, S. et al., Cigarette Smoking Induces An Increase In Oxidative DNA Damage, 8-hydroxydeoxyguanosine, in the Central Site of the Human Lung, Carcinogenesis, 18:1763-1766 (1997), hereby incorporated by reference to its entirety.

Another gene polymorphism that has been reported as a probable predictive marker for lung cancer susceptibility among smokers is the SUV39H2 histone methyltransferase. Eight single nucleotide polymorphisms (SNPs) of SUV39H2 were identified in Korean population and the identification of a SNP in the 3′UTR of SUV39H2 was associated with lung cancer susceptibility in the Korean population. See Yoon, K.-A. et al., Novel Polymorphisms in the SUV39H2 Histone Methyltransferase and Risk of Lung Cancer, Carcinogenesis, Vol. 27(11):2217-2222 (2006), hereby incorporated by reference to its entirety

Immuno-Markers from Early Lung Cancer Detection Test (EarlyCDT®-Lung)

Patients with lung cancer can mount a humoral immune response to the tumor cells or tumor-derived or associated antigens (TAAs) leading to the production of autoantibodies (Aabs). The presence of these autoantibodies in lung cancer patients and their levels as measured accordingly can aid in early cancer detection and selection and monitoring of treatment. An autoantibody or immunobiomarker test for early detection of lung cancer that measure AAB against a panel of TAAs is currently available and used in the U.S. The EarlyCDT®-Lung test has been approved by Clinical Laboratory Improvement Amendments (CLIA) and as such had met the criteria of an accepted standard, having been through both technical and clinical validation. See Lam, S. et al., EarlyCDT®-Lung: An Immunobiomarker Test as an Aid to Early Detection of Lung Cancer, Cancer Prev. Res., 4(7):1126-1134 (2011); Boyle, P. et al., Clinical Validation of an Autoantibody Test for Lung Cancer, Ann. Oncol., 22:383-389 (2011); Murray, A. et al., Technical Validation of an Autoantibody Test for Lung Cancer, Ann. Oncol. 21:1687-1693 (2010), both of which are hereby incorporated by reference to their entirety. The initial EarlyCDT®-Lung test includes a panel of AAbs to sic tumor-associated antigens (p53, NY-ESO-1, annexin 1, CAGE, GBU4-5, and SOX-2). More recently, an improved EarlyCDT®-Lung test includes an additional two AAbs (MAGE4 and HuD) and the removal of annexin 1 from the panel. The improved seven TAA EarlyCDT®-Lung test has a reported specificity of 93% and a sensitivity of 41% for the detection of lung cancer. See Chapman, C. J. et al., The EarlyCDT®-Lung Test: Improved Early Clinical Utility Through Additional Autoantibody Assays, Tumor Biol. 33(5):1319-1326, (2012), hereby incorporated by reference to its entirety. Recently, the application of a high throughput cloning and expression methods have been developed and aided in identifying five potential immunobiomarkers for early lung cancer detection. These additional immunobiomarkers include alpha-enolase, MGP1 (myc-binding protein 1), cytokeratin 8, cytokeratin 20 and L-myc. See Macdonald, I. K. et al., Application of a High Throughput Method of Biomarker Discovery to Improvement of the EarlyCDT®-Lung Test, PLoS One. 7(12):e51002 (2012), hereby incorporated by reference to its entirety.

Cigarette smoking cessation was followed by a marked increase in plasma levels of several antioxidant micronutrients and improves resistance towards oxidative challenge. See Br. J. Nutr., 90:147-150 (2003), hereby incorporated by reference to its entirety. Smoking cessation can be achieved with or without assistance from healthcare professionals or the use of medications.

### Biomarker Panel

A “biomarker panel” disclosed herein may be combined with measurements of other biomarkers and clinical parameters to assess patient compliance with a smoking cessation program.

Also disclosed are behavior modification programs, according to the embodiment of the invention, for assessing a subject's compliance with a smoking cessation program particularly through the use of biomarker panels described hereinabove. A large number of biomarkers are known for a variety of cardiovascular, cancer and other tobacco-health related conditions. The biomarker panels disclosed herein allow the practitioner to directly identify whether the subject is partially or fully in compliance or fail to comply with the smoking cessation program and to use when implementing the disclosed behavior modification programs.

The term “biomolecular marker,” “biomarker” or “marker” (also sometimes referred to herein as a “target analyte,” “target species” or “target sequence”) refers to a molecule whose measurement provides information as to the state of a subject. In various exemplary embodiments, the biomarker is used to assess a pathological state and can be detected in any biological sample of the subject.

As described herein, a biomarker may be, for example, a small molecule, an analyte or target analyte, a lipid (including glycolipids), a carbohydrate, a nucleic acid, a protein, any derivative thereof or a combination of these molecules, with proteins and nucleic acids finding particular use in the invention. As will be appreciated by those in the art, a large number of analytes may be detected using the present methods; basically, any biomarker for which a binding ligand, described below, may be made may be detected using the methods of the invention.

In various exemplary embodiments, the biomarker panel comprises one or more carcinogen biomarkers, wherein the one or more carcinogen biomarker includes 4-(methylnitrosoamino)-1-(3-pyridyl)-1-butanol (NNAL) and at least one carcinogen biomarkers include MHBMA, HPMA, HBMA, SPMA, HEMA, 1-HOP, NNAL, NNK, polycystic aromatic hydrocarbon-DNA adducts (PAH-DNA), 4-ABP-hemoglobin adducts, benzene and benzene metabolites, and OGG1 activity.

In various exemplary embodiments, the biomarker panel comprises one or more antioxidants, e.g., vitamin A, vitamin C, vitamin E, lutein, lycopene, and β-carotene.

In various exemplary embodiments, the biomarker panel comprises one or more oxidative-stress biomarkers, e.g., malondialdehyde (MDA), oxidized glutathione (GSSG), reduced glutathione (GSH), and GSSG/GSH ratio.

In various exemplary embodiments, the biomarker panel comprises one or more cardiovascular risk biomarkers, e.g., low density lipoprotein particle number (LDL-P), LDL-cholesterol (LDL-C), apolipoprotein A-1 (ApoA-1), apolipoprotein B (ApoB), triglyceride, high density lipoprotein particle number (HDL-P), high density lipoprotein-cholesterol (HDL-C), high sensitivity C-reactive protein (hs-CRP), remnant-like lipoproteins (RLPs), RLP-c (cholesterol measures), lipoprotein A (apoA-I, Lp(a) or HDL), HDL2, ApoB:ApoA-1 ratio, ApoB, ApoB48, ApoE, ApoC, Lp(a) mass, Lp(a) cholesterol, large VLDL-P, small LDL-P, large HDL-P, VLDL-size, LDL size, HDL size, LP-IR score, and subclasses, genetic variants, fragments and complexes thereof.

In various exemplary embodiments, the biomarker panel comprises one or more analytes associated with insulin resistance, glycemia and/or beta cell dysfunction, e.g., glucose, insulin, hemoglobin (Hb) A1c, fructosamine, mannose, D-mannose, mannose-binding lectin, 1,5-anhydroglucitol (1,5 AG), glycation gap (glycosylation gap), serum amylase, anti-GAD antibody, c-peptide, intact pro-insulin, leptin, adiponectin, ferritin, free fatty acids, lipoprotein-associated phospholipase A2 (Lp-PLA2), fibrinogen, myeloperoxidase, cystatin C, homocysteine, F2-isoprostanes, α-hydroxybutyrate (AHB), linoleoyl glycerophosphocholine (GPC), oleic acid, analytes associated with IR score, analytes associated with HOMA (Homeostasis Model Assessment) IR score, analytes associated with CLIX score, gamma-glutamic transferase (GGT), uric acid, vitamin B12, homocysteine, 25-hydroxyvitamin D, TSH, earned glomerular filtration rate, and serum creatinine.

In various exemplary embodiments, the biomarker panel comprises a plurality of immuno-markers from an early lung cancer detection test (EarlyCDT®) or one or more polymorphic genes involved in the risk of development of cancer or detection of presence of cancer. Examples of single nucleotide polymorphism (SNP) may include variegation 3-9 homolog 2 (SUV39H2) polymorphism and CRP gene polymorphism

The selection of the suitable biomarker panel for use to assess the patient compliance will depend upon the preference of the treating physician, the smoking habits of the subject being treated and other aspects relating to the patient and his or her tobacco use that will be appreciated by the treating physician. In some cases, for example, the physician may employ a biomarker panel that includes the first analyte-binding ligand, the second analyte-binding ligand and several other biomarkers including carcinogen biomarkers, antioxidants, oxidative-stress biomarkers, analytes associated with insulin resistance, glycemic control and/or beta cell dysfunction, immuno-markers and polymorphic genes. In other cases, for example, the physician may utilize a biomarker panel that includes the first analyte-binding ligand, the second analyte-binding ligand and only two or three of additional biomarkers.

A biomarker can also be a clinical parameter, although in some embodiments, the biomarker is not included in the definition of “biomarker”. The term “clinical parameter” refers to all non-sample or non-analyte biomarkers of subject health status or other characteristics, such as, without limitation, age, ethnicity, gender, diastolic blood pressure and systolic blood pressure, family history, height, weight, waist and hip circumference, body-mass index, resting heart rate, β cell function, macrovascular function, microvascular function, atherogenic index, blood pressure, low-density lipoprotein/high-density lipoprotein ratio, intima-media thickness, and UKPDS risk score. Other clinical parameters are disclosed in U.S. Patent Application No./20080057590, hereby incorporated by reference to its entirety.

Biomarkers generally can be measured and detected through a variety of assays, methods and detection systems known to one of skill in the art. The term “measuring,” “detecting,” or “taking a measurement” refers to a quantitative or qualitative determination of a property or characteristic of an entity, e.g., quantifying the amount, concentration, level or value or the activity level of a molecule. The term “concentration,” “value,” “amount” or “level” can refer to an absolute or relative quantity. Measuring a molecule may also include determining the absence or presence of the molecule. A measurement may refer to one observation under a set of conditions or an equally- or differently-weighted average of a plurality of observations under the same set of conditions. Thus, in various embodiments, a measurement of the concentration, value, amount or level of a biomarker is derived from one observation of the concentration, and in various embodiments, a measurement of a biomarker is derived from an equally- or differently-weighted average of a plurality of observations of the concentration. In various embodiments, measuring a biomarker panel comprises measuring the concentrations, values, amounts or levels of each member of the biomarker panel in a sample.

Various methods for measuring biomarkers, include but are not limited to, refractive index spectroscopy (RI), ultra-violet spectroscopy (UV), fluorescence analysis, radiochemical analysis, near-infrared spectroscopy (near-IR), infrared (IR) spectroscopy, nuclear magnetic resonance spectroscopy (NMR), light scattering analysis (LS), mass spectrometry (MS), pyrolysis mass spectrometry, nephelometry, dispersive Raman spectroscopy, gas chromatography (GC), liquid chromatography (LC), gas chromatography combined with mass spectrometry (GC/MS), liquid chromatography combined with mass spectrometry (LC/MS), matrix-assisted laser desorption ionization-time of flight (MALDI-TOF) combined with mass spectrometry, ion spray spectroscopy combined with mass spectrometry, capillary electrophoresis, colorimetry and surface plasmon resonance (such as according to systems provided by Biacore Life Sciences). See also WO/2004/056456 and WO/2004/088309, both of which are hereby incorporated by reference to their entirety. In this regard, biomarkers can be measured using the above-mentioned detection methods, or other methods known to the skilled artisan. Other biomarkers can be similarly detected using reagents that are specifically designed or tailored to detect them.

The biomarkers used in the panels of the invention can be detected either as proteins or as nucleic acids (e.g. mRNA or cDNA transcripts) in any combination. The protein form of a biomarker can also be measured. As will be appreciated by those in the art, protein assays may be done using standard techniques such as ELISA assays. Also, the nucleic acid form of a biomarker (e.g., the corresponding mRNA) can also be measured. As an example, one or more biomarkers from a particular panel can be measured using a protein assay and one or more biomarkers from the same panel can be measured using a nucleic acid assay.

The term “analyte” refers to any molecule or fragment thereof to be detected or measured including, but not limited to, cotinine and/or anabasine, or its fragments thereof to be detected or measured.

The term “ligand” as used herein refers to a molecule that binds to an epitope or binding site. The term, as used herein, includes antibodies, proteins, peptides, polypeptides, amino acids, nucleic acids, carbohydrates, sugars, lipids, organic molecules, polymers, putative therapeutic agents, and the like.

The terms “binding agent,” “binding ligand,” “capture binding ligand,” “capture probe” or grammatical equivalents are used interchangeably with reference to a compound or large molecule that is used to detect the presence of or to quantify, relatively or absolutely, a target analyte, target species or target sequence (all used interchangeably) corresponding to a suitable biomarker. Generally, the binding agent or capture probe allows the attachment of a target species or target sequence to a solid support for the purposes of detection as further described herein. Attachment of the target species to the binding agent may be direct or indirect. As will be appreciated by those in the art, the composition of the binding ligand will depend on the composition of the biomarker. Binding ligands for a wide variety of biomarkers are known or can be readily found using known techniques. For example, when the biomarker is a protein, the binding ligands include proteins (particularly including antibodies or fragments thereof (FAbs, etc.) as discussed further below) or small molecules. The binding ligand may also have cross-reactivity with proteins of other species. Antigen-antibody pairs, receptor-ligands, and carbohydrates and their binding partners are also suitable analyte-binding ligand pairs. In various embodiments, the binding ligand may be a nucleic acid.

In several exemplary embodiments, the target analyte or target species includes cotinine and/or anabasine or an antigen-binding fragment thereof and at least two or more of the following biological markers, namely: one or more carcinogen markers, one or more anti-oxidants, one or more oxidative-stress biomarkers, one or more cardiovascular risk biomarkers, one or more analytes associated with insulin resistance, glycemic control and/or beta cell dysfunction, one or more immuno-markers from an early lung cancer detection test (EarlyCDT®); and one or more polymorphic genes involves in the risk of development or detection of presence of cancer. As will be appreciated by those in the art, the composition of the binding agent will depend on the composition of the appropriate or suitable biomarker (s).

As further defined below, a ligand that “specifically binds” or “selectively binds” or is “selective for” a biomarker means that the ligand binds the biomarker with specificity sufficient to differentiate between the biomarker and other components or contaminants of the sample.

Capture binding ligands that are useful in the present invention may be “selective” for, “specifically bind” or “selectively bind” their target, such as a protein. Typically, specific or selective binding can be distinguished from non-specific or non-selective binding when the dissociation constant (KD) is less than about 1×10⁻⁵M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

In various exemplary embodiments, the capture binding ligand is an antibody. These embodiments are particularly useful for the detection of the protein form of a biomarker.

The terms “antigen,” “immunogen,” “antigenic,” “immunogenic,” “antigenically active,” “immunologic,” and “immunologically active” when made in reference to a molecule, refer to any substance that is capable of inducing a specific humoral immune response (including eliciting a soluble antibody response) and/or cell-mediated immune response (including eliciting a CTL response). To elicit antibody production, in one embodiment, small molecules, or haptens, may be conjugated to ovalbumin (OVA), keyhole limpet hemocyanin (KLH), bovine serum albumin (BSA) or fused to glutathione-S-transferase (GST).

In one embodiment, the antigen comprises an epitope. The terms “epitope” and “antigenic determinant” refer to a structure on an antigen, which interacts with the binding site of an antibody or T cell receptor as a result of molecular complementarity. An epitope may compete with the intact antigen, from which it is derived, for binding to an antibody.

As used herein the terms “portion” and “fragment” when made in reference to a nucleic acid sequence or protein sequence refer to a piece of that sequence that may range in size from 2 contiguous nucleotides and amino acids, respectively, to the entire sequence minus one nucleotide and amino acid, respectively.

Detection of a target species in some embodiments utilizes a “label” or “detectable marker” (as described below) that can be incorporated in a variety of ways. Thus, in various embodiments, the “binding agent,” “binding ligand” or “capture binding ligand comprises a “label” or a “detectable marker.” In one embodiment, the target species (or target analyte or target sequence) is labeled; binding of the target species thus provides the label at the surface of the solid support.

When using a label, a sandwich format can be utilized, in which target species are unlabeled. For example. a “capture” or “anchor” binding ligand is attached to the detection surface as described herein, and a soluble binding ligand (frequently referred to herein as a “signaling probe,” “label probe” or “soluble capture ligand”) binds independently to the target species and either directly or indirectly comprises at least one label or detectable marker.

By “label” or “labeled” herein is meant that a compound has at least one molecule, element, isotope or chemical compound attached to enable the detection of the compound. In general, labels fall into four classes: a) isotopic labels, which may be radioactive or heavy isotopes; b) magnetic, electrical, thermal; c) colored, fluorescent, chemiluminescent or luminescent dyes; and d) enzymes; although labels include particles such as magnetic particles as well. Suitable dyes for use in the present application include, but are not limited to, fluorescent lanthanide complexes, including those of Europium and Terbium, fluorescein isothiocyanate, biotin, rhodamine, tetramethylrhodamine, eosin, erythrosin, coumarin, methyl-coumarins, pyrene, malachite green, stilbene, Lucifer Yellow, Cascade Blue, Texas Red, Alexa dyes, and the like.

A secondary detectable label can also be employed. A secondary label is one that is indirectly detected; for example, a secondary label can bind or react with a primary label for detection, can act on an additional product to generate a primary label (e.g., enzymes), or may allow the separation of the compound comprising the secondary label from unlabeled materials, etc. Secondary labels include, but are not limited to, one of a binding partner pair; chemically modifiable moieties; nuclease inhibitors, enzymes such as β-galactosidase, horseradish peroxidase, alkaline phosphatases, luciferases, etc. Secondary labels can also include additional labels.

The secondary label can also be a binding partner pair. For example, the label may be a hapten or antigen, which will bind its binding partner. For example, suitable binding partner pairs include, but are not limited to: antigens (such as proteins (including peptides)) and antibodies (including fragments thereof (Fabs, etc.); proteins and small molecules, including biotin/streptavidin; enzymes and substrates or inhibitors; other protein-protein interacting pairs; receptor-ligands; and carbohydrates and their binding partners.

In the sandwich format, an enzyme can serve as the secondary label, bound to the soluble capture ligand. Of particular use is the use of horseradish peroxidase, which when combined with 3,3′,5,5′-tetramethylbenzidine (TMB) forms a colored precipitate which is then detected. In some cases, the soluble capture ligand comprises biotin, which is then bound to a enzyme-streptavidin complex and forms a colored precipitate with the addition of TMB.

The label or detectable marker can be a conjugated enzyme (for example, horseradish peroxidase). This system relies on detecting the precipitation of a reaction product or on a change in, for example, electronic properties for detection. It is also possible that none of the compounds contains a label.

Those skilled in the art will be familiar with numerous additional immunoassay formats and variations thereof which are useful for detecting proteins or antibodies. Examples of suitable immunoassays include immunoblotting, immunofluorescence methods, immunoprecipitation, chemiluminescence methods, electrochemiluminescence (ECL) or enzyme-linked immunoassays.

The term “antibody” refers to an immunoglobulin (e.g., IgG, IgM, IgA, IgE, IgD, etc.). The basic functional unit of each antibody is an immunoglobulin (Ig) monomer (containing only one immunoglobulin (“Ig”) unit). Included within this definition are polyclonal antibody, monoclonal antibody, single chain antibody, humanized antibody and chimeric antibody.

The variable part of an antibody is its “V domain” (also referred to as “variable region”), and the constant part is its “C domain” (also referred to as “constant region”) such as the κ, λ, α, γ, δ, ε and μ constant regions. The “variable domain” is also referred to as the “FV region” and is the most important region for binding to antigens. More specifically, variable loops, three each on the light (VL) and heavy (VH) chains are responsible for binding to the antigen. These loops are referred to as the “complementarity determining regions” (“CDRs” and “idiotypes.”

The immunoglobulin (Ig) monomer of an antibody is a “Y”-shaped molecule that contains four polypeptide chains: two light chains and two heavy chains, joined by disulfide bridges.

Light chains are classified as either λ or κ. A light chain has two successive domains: one constant domain (“CL”) and one variable domain (VL). The variable domain, VL, is different in each type of antibody and is the active portion of the molecule that binds with the specific antigen. The approximate length of a light chain is 211 to 217 amino acids. Each heavy chain has two regions, the constant region and the variable region. There are five types of mammalian Ig heavy denoted as α, δ, ε, γ, and μ. The type of heavy chain present defines the class of antibody; these chains are found in IgA, IgD, IgE, IgG, and IgM antibodies, respectively. Distinct heavy chains differ in size and composition; γ and γ. contain approximately 450 amino acids, while μ and ε have approximately 550 amino acids. Each heavy chain has two regions, the constant region (“CH”) and the variable (“VH”) region. The constant region (CH) is identical in all antibodies of the same isotype, but differs in antibodies of different isotypes. Heavy chains α and ε. have a constant region composed of three tandem (in a line) Ig domains, and a hinge region for added flexibility. Heavy chains μ and ε have a constant region composed of four immunoglobulin domains. The variable region (VH) of the heavy chain differs in antibodies produced by different B cells, but is the same for all antibodies produced by a single B cell or B cell clone. The variable region of each heavy chain is approximately 110 amino acids long.

“Enzyme immunoassay” and “EIA” are interchangeably used to refer to a method for detecting and/or determining the level of an antibody or an antigen in a sample. The antibody or antigen is conjugated to an enzyme, and the antibody and antigen are contacted under conditions for binding of the antibody to the antigen. An enzymatic substrate is added that the enzyme can convert to a detectable signal (e.g., chromogenic, fluorogenic, electrochemiluminescent, etc.). The level of antibody, or antigen, that is linked to the enzyme is quantified by measuring the level of the detectable signal. Enzyme immunoassays are exemplified by, but not limited to, an “enzyme-linked immunosorbent assay” (“ELISA”). Performing an ELISA involves at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “sandwich” ELISA). After the antigen is immobilized, the detection antibody is added, forming a complex with the antigen. The detection antibody can be covalently linked to an enzyme, or can itself be detected by a secondary antibody that is linked to an enzyme through bio-conjugation. Between each step the plate is typically washed with a mild detergent solution to remove any proteins or antibodies that are not specifically bound. After the final wash step the plate is developed by adding an enzymatic substrate to produce a detectable signal, which indicates the quantity of antigen in the sample.

In general, immunoassays may be homogeneous assays or heterogeneous assays. In a homogeneous assay the immunological reaction usually involves the specific antibody (e.g., anti-biomarker protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays include immunoblotting, immunofluorescence methods, immunoprecipitation, chemiluminescence methods, electrochemiluminescence (ECL) or enzyme-linked immunoassays.

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

The “biomolecular marker,” “biomarker” or “marker” (also sometimes referred to herein as a “target analyte,” “target species” or “target sequence”) as described above can be setected in any biological sample. A “biological sample” encompasses a variety of sample types obtained from an individual including, without limitation, blood and other liquid samples of biological origin, solid tissue samples such as a biopsy specimen or tissue cultures or cells derived therefrom and the progeny thereof. A biological also includes samples that have been manipulated in any way after their procurement, such as by treatment with reagents, solubilization, or enrichment for certain components. The term “biological sample” encompasses a clinical sample or a blood component, and also includes cells in culture, cell supernatants, cell lysates, serum, plasma, biological fluid, urine, tissue homogenates and tissue samples.

The term “correlating” generally refers to determining a relationship between one type of data with another or with a state. In one embodiment, correlating the measurement with disease comprises comparing the measurement with a reference biomarker profile or some other reference value. This can also include determining whether the subject is currently in a state of disease.

The quantity or activity measurements of a biomarker panel can be compared to a reference value. Differences in the measurements of biomarkers in the subject sample compared to the reference value are then identified. In exemplary embodiments, the reference value is given by a risk category as described further below.

The reference value can be a baseline value. A baseline value is a composite sample of an effective amount of biomarkers from one or more subjects who are non-smokers or passive smokers, who do not have a cardiovascular disease or cancer, who are asymptomatic for a disease or who have a certain level of a disease. A baseline value can also comprise the amounts of biomarkers in a sample derived from a subject who has shown an improvement in risk factors of a disease as a result of treatments or therapies. In these embodiments, to make comparisons to the subject-derived sample, the amounts of biomarkers are similarly calculated.

A reference value can also comprise the amounts of biomarkers derived from subjects who have a disease confirmed by an invasive or non-invasive technique, or are at high risk for developing a disease. Often times, data from reference values is available through publicly available database or publications on the subject matter. Optionally, subjects identified as having a disease, or being at increased risk of developing a disease are chosen to receive a therapeutic regimen to slow the progression of a disease, or decrease or prevent the risk of developing a disease. A disease is considered to be progressive (or, alternatively, the treatment does not prevent progression) if the amount of biomarker changes over time relative to the reference value, whereas a disease is not progressive if the amount of biomarkers remains constant over time (relative to the reference population, or “constant” as used herein). The term “constant” as used in the context of the present invention is construed to include changes over time with respect to the reference value.

The biomarkers can be used to generate a “reference biomarker profile” of those subjects who do not have a disease according to a certain threshold, are not at risk of having a disease or would not be expected to develop a disease. The biomarkers disclosed herein can also be used to generate a “subject biomarker profile” taken from subjects who have a disease or are at risk for having a disease. The subject biomarker profiles can be compared to a reference biomarker profile to diagnose or identify subjects at risk for developing a disease, to monitor the progression of disease, as well as the rate of progression of disease, and to monitor the effectiveness of disease treatment modalities.

The biomarker panels can be used by a practitioner or treating physician to determine and effect appropriate therapies with respect to a subject given the disease status indicated by measurements of the biomarkers in a sample from the subject. Thus, in one aspect, the invention provides a behavioral modification program for assessing a subject's compliance with a smoking cessation program that comprises measuring one or more biological samples with a set of biomarkers from the biomarker panels of the invention, assaying the levels of the set of biomarkers from the one or more biological samples of the subject; comparing the levels of the set of biomarkers obtained in the assessment step with reference levels of each corresponding set of reference biomarkers; and determining whether the patient is in compliance with the smoking cessation program based on the comparison. The behavioral modification program further includes effectuating a therapy guidance based on the determination obtained. The values or levels of the biomarkers of the biomarker panel may increase or decrease according to the values described herein or may stay the same in response to the therapy. The behavior modification program may be further modified or maintained based on the determination step.

In one embodiment, the therapy guidance may include drug therapy, recommendations on making or maintaining lifestyle choices based on the determination in step and reporting to the patient the cardiovascular and/or cancer risk based on the determination.

Lifestyle choices that can be implemented based on the patient's exposure to cigarette smoke, second hand smoke or cardiovascular and cancer risk profiles and can involve changes in diet, changes in exercise, reducing or eliminating smoking, or a combination thereof. As will be understood by the subject's treating physician and other medical professionals, the lifestyle choice is one that will lower risk for developing or having a cardiovascular disease, cancer or other health disorders or decrease the severity of the disease (see Eriksson et al., “A 3-Year Randomized Trial of Lifestyle Intervention for Cardiovascular Risk Reduction in the Primary Care Setting: The Swedish Bjorknas Study,” PLOS 4(4):e5195 (2009); Omish et al., “Intensive Lifestyle Changes for Reversal of Coronary Heart Disease,” JAMA 220(23): 2001-2007 (1998); and Wister et al., “One-year Follow-up of a Therapeutic Lifestyle Intervention Targeting Cardiovascular Disease Risk,” CMAJ 177(8):859-865 (2007), all of which are hereby incorporated by reference in their entirety).

The therapy guidance may include nicotine replacement therapy, drug prescription therapy, nutritional therapy and psychological counseling. Examples of nicotine replacement therapy may include gums, lozenges and transdermal nicotine-delivery patches.

Examples of drugs and nutrition for treating a subject undergoing the smoking cessation regimen may include bupropion (Zyban®), varinicline (Chantix®), a statin drug, niacin, fibrates, dietary supplement with fish oils, cholesterol absorption inhibitors, cholesterol-sequestering resins, Lovastatin/ERN (Advicor®), Simvastatin/ERN (SimCor®), Ezetimibe/Simvastatin (Vytorin®), anti-hypertensive drugs, and blood-glucose-lowering drugs.

The terms “therapy” and “treatment” may be used interchangeably. In certain embodiments, the therapy can be selected from, without limitation, initiating therapy, continuing therapy, modifying therapy or ending therapy. A therapy also includes any prophylactic measures that may be taken to prevent disease or the development of additional diseases.

Measurement of biomarker concentrations allows the health care provider to monitor patient's compliance and recovery to smoking cessation program and to provide therapeutic intervention and risk management. The effectiveness of a smoking cessation program and therapeutic intervention can be monitored by detecting one or more biomarkers in an effective amount from samples obtained from a subject over time and comparing the amount of biomarkers detected. For example, a first sample can be obtained prior to the subject's compliance to smoking cessation program or receiving treatment and one or more subsequent samples can be taken after or during the smoking cessation program or treatment of the subject. Changes in biomarker values or levels across the samples may provide an indication as to the success of the smoking cessation program and effectiveness of the therapy.

If a given smoker were tested for biomarkers of cardiovascular risk and cancer risk before smoking cessation, and on subsequent measurements the patient's risk profiles improved with successful cessation, the act of counseling the patient with the improvements in their risk profile would comprise a powerful tool for positive reinforcement of the quitting behavior. Conversely, if a given patient was non-compliant and testing also revealed cardiovascular disease risk and a genetic polymorphism predisposing the patient to a higher risk of developing cancer, the healthcare provider could use this information to change the patient's therapies, and use the results of the tests to counsel the patient on their elevated risk status. In this example, advising the patient that they remain at-risk for cardiovascular disease and cancer comprises negative reinforcement of the smoking behavior to encourage compliance.

According to B. F. Skinner (Skinner, B. F., Science and Human Behavior, 1953, hereby incorporated by reference to its entirety), operant conditioning refers to a process by which behavior operates under the environment and is strengthened by its consequences, which are called reinforcers. This can occur through both positive and negative reinforcement. Positive reinforcement refers to presenting or adding a stimulus to the environment to increase the probability of a behavior. Negative reinforcement refers to removing a stimulus from the environment to increase a response (Skinner, B. F., 1953). In dependent smokers, nicotine serves as a reinforcer for, or increases, smoking behavior because of pleasurable feelings the smoker experiences (positive reinforcement) and through the avoidance of withdrawal symptoms (negative reinforcement). See Lujic, C. et al., Psychobiological Theories of Smoking and Smoking Motivation, Eur. Psychologist, 10(1), 1-24 (2005) and Schare, M. L. & Konstas, D. (2008) Self-Help Therapies for Cigarette Smoking Cessation, In Watkins, P. L. & Clum, G. A. (Eds.), Handbook of Self-Help Therapies, Lawrence-Earlbaum Publishers, Inc., all of which are hereby incorporated by reference to its entirety).

Cigarette smoking appears to be a habit because it is a learned, repetitive behavior that is influenced by learning and reinforcement. Smoking behaviors are so over learned that smokers may light a cigarette and forget about it. See Seidman et al., Women and Smoking Towards the Millennium, Isr. Med. Assoc. J., 1(3):215-217 (1999)), hereby incorporated by reference to its entirety. These behaviors, which are involved in seeking, lighting and smoking cigarettes, become conditioned through both positive and negative reinforcements. See Kozlowski, L. T. et al., Cigarettes, Nicotine and Health: A Behavioral Approach, Sage Productions, Inc., London, hereby incorporated by reference to its entirety.

In addition to the above, a “positive reinforcement” refers to the behavior that produces a rewarding effect that would not have occurred otherwise. See Eissenberg, T., Measuring the Emergence of Tobacco Dependence: the Contribution of Negative Reinforcement Models, Addiction, 99(S1):5-29 (2004) and Kozlowski, L. T. et al., Cigarettes, Nicotine and Health: A Behavioral Approach, Sage Productions, Inc., London, both of which are hereby incorporated by reference to their entirety. Drugs may serve as positive reinforcers meaning that they produce repetition and strengthen behaviors that lead to further administration. See The Health Consequences of Smoking: Nicotine Addiction: A Report of the Surgeon General, U.S. Department of Health and Human Services (1988), hereby incorporated by reference to its entirety.

Negative reinforcement, on the other hand, refers to a behavior that is performed to avoid an aversive event or terminate an unpleasant state. Self-administration leads to termination or avoidance of withdrawal symptoms. The onset of drug dependence can occur when the avoidance of withdrawal begins to motivate self-administration and drug taking behaviors. See Eissenberg, T., Measuring the Emergence of Tobacco Dependence: the Contribution of Negative Reinforcement Models, Addiction, 99(S1):5-29 (2004), hereby incorporated by reference to its entirety.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A biomarker panel for assessing patient compliance with a smoking cessation program, the biomarker panel comprising:
a) a first analyte-binding ligand that specifically binds to cotinine, or an antigen-binding fragment thereof;
b) a second analyte-binding ligand that specifically binds to anabasine, or an antigen-binding fragment thereof; and
c) at least two or more of the following biomarkers:
i. one or more carcinogen biomarkers, wherein the one or more carcinogen biomarker includes 4-(methylnitrosoamino)-1-(3-pyridyl)-1-butanol (NNAL) and at least one carcinogen biomarkers selected from the group consisting of MHBMA, HPMA, HBMA, SPMA, HEMA, 1-HOP, NNAL, NNK, polycystic aromatic hydrocarbon-DNA adducts (PAH-DNA), 4-ABP-hemoglobin adducts, benzene and benzene metabolites, and OGG1 activity;
ii. one or more antioxidants selected from the group consisting of vitamin A, vitamin C, vitamin E, lutein, lycopene, and β-carotene;
iii. one or more oxidative-stress biomarkers selected from the group consisting of malondialdehyde (MDA), oxidized glutathione (GSSG), reduced glutathione (GSH), and GSSG/GSH ratio;
iv. one or more cardiovascular risk biomarkers;
v. one or more analytes associated with insulin resistance, glycemic control, and/or beta cell dysfunction;
vi. a plurality of immuno-markers from an early lung cancer detection test (EarlyCDT®); and
vii. one or more polymorphic genes involved in the risk of development of cancer or detection of presence of cancer.

2. The biomarker panel of claim 1, wherein the one or more cardiovascular risk biomarkers is selected from the group consisting of low density lipoprotein particle number (LDL-P), LDL-cholesterol (LDL-C), apolipoprotein A-1 (ApoA-1), apolipoprotein B (ApoB), triglyceride, high density lipoprotein particle number (HDL-P), high density lipoprotein-cholesterol (HDL-C), high sensitivity C-reactive protein (hs-CRP), remnant-like lipoproteins (RLPs), RLP-c (cholesterol measures), lipoprotein A (apoA-I, Lp(a) or HDL), HDL2, ApoB:ApoA-1 ratio, ApoB, ApoB48, ApoE, ApoC, Lp(a) mass, Lp(a) cholesterol, large VLDL-P, small LDL-P, large HDL-P, VLDL-size, LDL size, HDL size, LP-IR score, and subclasses, genetic variants, fragments and complexes thereof.

3. The biomarker panel of claim 1, wherein the one or more analytes associated with insulin resistance, glycemia and/or beta cell dysfunction is selected from the group consisting of glucose, insulin, hemoglobin (Hb) A1c, fructosamine, mannose, D-mannose, mannose-binding lectin, 1,5-anhydroglucitol (1,5 AG), glycation gap (glycosylation gap), serum amylase, anti-GAD antibody, c-peptide, intact pro-insulin, leptin, adiponectin, ferritin, free fatty acids, lipoprotein-associated phospholipase A2 (Lp-PLA2), fibrinogen, myeloperoxidase, cystatin C, homocysteine, F2-isoprostanes, α-hydroxybutyrate (AHB), linoleoyl glycerophosphocholine (GPC), oleic acid, analytes associated with IR score, analytes associated with HOMA (Homeostasis Model Assessment) IR score, analytes associated with CLIX score, gamma-glutamic transferase (GGT), uric acid, vitamin B12, homocysteine, 25-hydroxyvitamin D, TSH, earned glomerular filtration rate, and serum creatinine.

4. The biomarker panel of claim 1, wherein the one or more cancer polymorphic genes is selected from the group consisting of variegation 3-9 homolog 2 (SUV39H2) polymorphism, CRP gene polymorphism, and genetic polymorphisms for DNA repair enzymes.

5. The biomarker panel of claim 1, wherein the at least two or more of the biomarkers comprise the biomarkers in (i), (iv), (v), and (vi).

6. The biomarker panel of claim 1, wherein the at least two or more of the biomarkers comprise the biomarkers in (i), (iv), and (v).

7. The biomarker panel of claim 1, wherein the at least two or more of the biomarkers comprise the biomarkers in (i), (ii), (iii), and (v).

8. The biomarker panel of claim 1, wherein the at least two or more of the biomarkers comprise the biomarkers in (i), (ii), and (iii).

9. The biomarker panel of claim 1, wherein the first analyte-binding ligand or the second analyte-binding ligand comprises an antibody.

10. The biomarker panel of claim 1, wherein the first analyte-binding ligand or the second analyte-binding ligand binds specifically to cotinine or anabasine.

11. The biomarker panel of claim 1, wherein the first analyte-binding ligand or the second analyte-binding ligand further comprises a first soluble capture ligand or a second soluble capture ligand that binds specifically to cotinine or anabasine.

12. The biomarker panel of claim 11, wherein the first soluble capture ligand or the second soluble capture ligand comprises a detectable label selected from the group consisting of a radioisotope, a fluorescent or chemiluminescent compound, and an enzyme.

13. The biomarker panel of claim 12, wherein the detectable label is selected from the group consisting of fluorescein isothiocyanate, rhodamine, luciferin, biotin, alkaline phosphatase, β-galactosidase, and horseradish peroxidase.

14. The biomarker panel of claim 11, wherein the detectable label is quantified by immunoassay methods selected from the group consisting of competitive binding assay, direct and indirect sandwich assay, immunoprecipitation assay, immunohistochemistry, enzyme-linked immunosorbent assay (ELISA), fluorescence-activated cell sorting (FACS), and Western blot assay.

15. The biomarker panel of claim 11, wherein the detectable label is quantified by nuclear magnetic resonance (NMR), mass spectrometry (MS), high performance liquid chromatography (HPLC), gas liquid chromatography (GLC) or a combination thereof.

16. A behavior modification program for assessing a subject's compliance with a smoking cessation program, comprising:
a) providing one or more biological samples from the subject, the subject being supervised by a health care provider in the smoking cessation program;
b) contacting the one or more biological samples with a set of biomarkers from the biomarker panel of claim 1;
c) assaying the levels of the set of biomarkers from the one or more biological samples of the subject;
d) comparing the levels obtained in step (c) with reference levels of each corresponding set of reference biomarkers; and
e) determining whether the patient is in compliance with the smoking cessation program based on the comparison in step (d).

17. The behavior modification program of claim 16, further effectuating a therapy guidance based on the determination in step (e).

18. The behavior modification program of claim 16, further comprising modifying or maintaining the behavior modification program for the subject based on the determination in step (e).

19. The behavior modification program of claim 16, further comprising identifying the levels of the set of biomarkers in step (d) in the one or more biological samples as normal, increased or decreased.

20. The behavior modification program of claim 17, wherein the therapy guidance involves drug therapy, recommendations on making or maintaining lifestyle choices based on the determination in step (e) and reporting to the patient the cardiovascular and/or cancer risk based on the determination in step (e).

21. The behavior modification program of claim 20, wherein lifestyle choices involve changes in diet and nutrition, changes in exercise, smoking elimination and/or a combination thereof.

22. The behavior modification program of claim 17, wherein the therapy guidance further comprises nicotine replacement therapy; drug prescription therapy, nutritional therapy and psychological counseling.

23. The behavior modification program of claim 22, wherein the nicotine replacement therapy is selected from the group consisting of gums, lozenges and transdermal nicotine-delivery patches.

24. The behavior modification program of claim 22, wherein the drugs and nutrition for treating the subject are selected from the group consisting of bupropion (Zyban®), varinicline (Chantix®), a statin drug, niacin, fibrates, dietary supplement with fish oils, cholesterol absorption inhibitors, cholesterol-sequestering resins, Lovastatin/ERN (Advicor®), Simvastatin/ERN (SimCor®), Ezetimibe/Simvastatin (Vytorin®), anti-hypertensive drugs, and blood-glucose-lowering drugs.

25. The behavior modification program of claim 16, wherein the biological sample is selected from the group consisting of a blood component, urine and saliva.

26. The behavior modification program of claim 16, wherein a baseline (pre-treatment) sample from the patient is analyzed and compared to one or more post-treatment samples to assess patient compliance with the smoking cessation program.

27. The behavior modification program of claim 16, wherein the levels of one or more cardiovascular risk biomarkers from the biomarker panel correlate the patient's smoking habits with an increased or decreased risk of a cardiovascular disease.

28. The behavior modification program of claim 16, wherein the levels of two or more carcinogen biomarkers from the biomarker panel correlate the patient's smoking habits with an increased or decreased risk of a cancer.

29. The behavior modification program of claim 16, wherein the levels of one or more antioxidants from the biomarker panel correlate the patient's smoking habits with an increased or decreased risk of a cardiovascular disease or a cancer.

30. The behavior modification program of claim 16, wherein the levels of one or more oxidative-stress markers from the biomarker panel correlate the patient's smoking habits with an increased or decreased risk of a cardiovascular disease or a cancer.

31. The behavior modification program of claim 16, wherein the levels of one or more analytes associated with insulin resistance, glycemia and/or beta cell dysfunction from the biomarker panel of claim 1 correlate the patient's smoking habits with an increased or decreased risk of a cardiovascular disease or a cancer.

32. The behavior modification program of claim 16, wherein the levels of the plurality of immuno-markers from an early lung cancer detection test (EarlyCDT®) and the levels of one or more cancer polymorphic genes correlate the patient's smoking habits with an increased or decreased risk of a cardiovascular disease or a cancer.

33. The behavior modification program of claim 16, wherein the behavior modification program provides positive or negative reinforcement to motivate patient compliance with the prescribed therapy guidance.

34. The behavior modification program of claim 16, wherein after-patient compliance with the smoking cessation program decreases the risk of morbidity and mortality by cardiovascular disease and cancer as a result of behavior modifications.

35. The behavior modification program of claim 16, wherein the steps (c), (d) and (e) are run repeatedly during the course of the smoking cessation program to assess patient's continued compliance and recovery.
